(19) United States
(12) Patent Application Publication
(10) Pub. No.: US 2023/0355930 A1
(43) Pub. Date: Nov. 9, 2023

(54) INTRAVENOUS IN FUSION SET AND ACCESSORY THEREFOR

(71) Applicant: Daniel Mark Holliday, Johannesburg (ZA)

(72) Inventor: Daniel Mark Holliday, Johannesburg (ZA)

(21) Appl. No.: 18/038,145

(22) PCT Filed: Nov. 26, 2021
(86) PCT No.: PCT/ZA2021/050066

§ 371 Date: May 22, 2023

(30) Foreign Application Priority Data
Nov. 26, 2020 (ZA) 2020/07368

(51) Int. Cl. A61M 25/02; A61M 5/14; A61M 39/08

(52) U.S. Cl. CPC A61M 25/02; A61M 5/1418; A61M 39/08

(57) ABSTRACT

This invention relates to an intravenous infusion set and accessory for an intravenous infusion. There is provided an accessory for securing an intravenous infusion set to at least part of a user’s forearm arm including primary engagement means (2) for engaging the accessory to a portion of a main intravenous line (3) and secondary engagement means 5 for simultaneously engaging the intravenous line (3) to another portion along the line (3). The primary engagement means (2) is displaceable along the line 3 such the position of the accessory (1) may be adjusted along the length of the line (3) and encloses the periphery of the line (3). The primary (2) and secondary (5) engagement means are proximate such that, operatively, when the secondary engagement means (5) engages the line (3), the line (3) forms a loop (6) which may be positioned to engage at least part of the user’s forearm.

### FIELD OF THE INVENTION

This invention relates to an intravenous infusion set and accessory for an intravenous infusion set and more specifically, but not exclusively to an intravenous infusion set and accessory for an intravenous infusion set which restrains the set from accidental removal from a patient.

### BACKGROUND TO THE INVENTION

Intravenous infusion sets, also referred to as intravenous infusion lines, are medical devices which are used in intravenous therapy. In the industry, the terms intravenous (IV) sets, IV lines, and IV therapy are often used as shorthand for reference.

In IV therapy, a bag filled with fluids and medication is suspended from a pole above a patient and connected to the patient with an IV set. An IV set typically includes a drip chamber, connected directly to the bag, on one end of the set and a peripheral venous catheter, which may be inserted into a peripheral vein of a patient, on the other end of the line. Between the catheter and the drip chamber is the main line (tube) of the IV set. Various accessories are typically pre-packaged along the IV set including various check valves, ports, clamps, and locks. The catheter is inserted into a vein of the patient and a cannula remains in the vein for administration of the fluids and medication in the bag.

A problem with traditional IV sets, when used on a patient, is that the cannula may be inadvertently pulled out of the patient’s vein when the patient moves about or rolls around while sleeping. Often the patient may be unaware that the cannula has pulled out and will not receive the necessary IV fluids and medication. This leads to uncertainty as to the dosing schedule of the patient. It also wastes valuable time of trained nurses or other medical staff to re-insert the IV set into the patient.

### OBJECT OF THE INVENTION

It is accordingly an object of this invention to provide an intravenous infusion set and accessory for an intravenous infusion set which, at least partially, alleviates the problems associated with the prior art or provides a useful alternative thereto.

### SUMMARY OF THE INVENTION

In accordance with the invention there is provided an accessory for securing an intravenous infusion set to at least part of a user’s forearm arm comprising:
primary engagement means for engaging the accessory to a portion of a main intravenous line of the set;
the primary engagement means being displaceable along the intravenous line; and
secondary engagement means for simultaneously engaging the intravenous line to another portion along the line;
the primary and secondary engagement means being proximate to one another such that, operatively, when the secondary engagement means engages the line, the line forms a loop; and
wherein the primary engagement means engages the line such that it is movable along the line and not removable from the line when in use.

The primary and secondary engagement means may frictionally engage the line.

The primary engagement means may be a tube.

The primary engagement means may be a sleeve.

The primary engagement means may be a ring.

The secondary engagement means may be a tube.

The secondary engagement means may be a sleeve.

The secondary engagement means may be a clip. The clip may form a snap-fit engagement with the tube.

The secondary engagement means may be an adhesive strip. The adhesive strip may include a writable portion such that the strip may be used as a label.

The secondary engagement means may include a tag for writing on or attaching a pre-printed label.

The secondary engagement means may be a shaped sheet of adhesive material. The shape sheet may be cross-shaped.

The primary and secondary engagement means may be parallel to each other and maintain the engaged portions of the lines parallel to each other.

The primary and secondary engagement means may be at an angle to each other to maintain an angle between the engaged portions of the line.

The primary and secondary engagement means may be integrally formed.

The primary and secondary engagement means may be pivotally attached to one another such that a hinged or pivotal connection is formed.

The secondary engagement means may include accessory attachment means, for attaching additional accessories to the accessory.

The secondary engagement means may be movable between an engaging position and a non-engaging position.

The primary or secondary engagement means may include adjustable line restricting means for adjustably restricting the flow of fluid through the engaged portion of the line.

In accordance with a second aspect of the invention there is provided an intravenous infusion set comprising:
a tubular main line;
a catheter engagement mechanism at one end of the main line;
a drip chamber at the other end of the main line;
a loop in the main line wherein the main line bends around and crosses itself to form the loop; and
an accessory which retains the loop in the main line such the line may be moved relative to the accessory to adjust a dimension of the loop;
wherein, in use, the loop may engage at least part of a patient’s arm such that movement of the line between the drip chamber and the loop will be imparted to the patients arm and prevents the movement from being imparted on the catheter engagement end of the IV set.

The IV set may be pre-packaged in packaging with the loop preformed therein.

The dimension may be the size or diameter of the loop.

The catheter engaging mechanism may be a cannula lock. The cannula lock may be a Luer lock.

The accessory may be in accordance with the accessory described above.

In accordance with a third aspect of the invention there is provided a method of setting up an intravenous set:
providing an IV set with a tubular main line, a distal end, and a proximal end;
forming a loop in the main line between the proximal and distal ends of the line;
engaging the loop to at least part of a patient’s arm; and
securing the loop to maintain the loop about the patient’s arm.

The step of securing the loop may include the step of providing an accessory along the main line which includes engagement means for maintaining the loop in the main line and about at least part of the patient’s arm.

The accessory may be in accordance with the accessory described above.

### BRIEF DESCRIPTION OF THE DRAWINGS

An embodiment of the invention is described below, by way of a non-limiting example only, and with reference to the accompanying drawing in which:

FIG. 1 is a schematic representation of a first embodiment of an accessory for an intravenous infusion set;

FIG. 2 is a schematic representation of the first embodiment of an accessory for an intravenous infusion set;

FIG. 3 is a schematic representation of the first embodiment of an accessory for an intravenous infusion set in use;

FIG. 4 is a schematic representation of a second embodiment of an accessory for an intravenous infusion set;

FIG. 5 is a schematic representation of a prepacked IV set;

FIG. 6 is a schematic representation of a third embodiment of an accessory for an intravenous infusion set;

FIG. 7 is a schematic representation of the third embodiment of an accessory for an intravenous infusion set;

FIG. 8 is a schematic representation of a fourth embodiment of an accessory for an intravenous infusion set;

FIG. 9 is a schematic representation of the fourth embodiment of an accessory for an intravenous infusion set;

FIG. 10 is a schematic representation of a fifth embodiment of an accessory for an intravenous infusion set;

FIG. 11 is a schematic representation of the fifth embodiment of an accessory for an intravenous infusion set;

FIG. 12 is a schematic representation of a sixth embodiment of an accessory for an intravenous infusion set;

FIG. 13 is a schematic representation of the sixth embodiment of an accessory for an intravenous infusion set;

FIG. 14 is a schematic representation of a seventh embodiment of an accessory for an intravenous infusion set;

FIG. 15 is a schematic representation of the seventh embodiment of an accessory for an intravenous infusion set;

FIG. 16 is a schematic representation of the seventh embodiment of an accessory for an intravenous infusion set in use;

FIG. 17 is a schematic representation of an eight embodiment of an accessory for an intravenous infusion set;

FIG. 18 is a schematic representation of the eight embodiment of an accessory for an intravenous infusion set;

FIG. 19 is a schematic representation of the eight embodiment of an accessory for an intravenous infusion set in use;

FIG. 20 is a schematic representation of a ninth embodiment of an accessory for an intravenous infusion set;

FIG. 21 is a schematic representation of a tenth embodiment of an accessory for an intravenous infusion set;

FIG. 22 is a schematic representation of the tenth embodiment of an accessory for an intravenous infusion set;

FIG. 23 is a schematic representation of an eleventh embodiment of an accessory for an intravenous infusion set;

FIG. 24 is a schematic representation of the eleventh embodiment of an accessory for an intravenous infusion set;

FIG. 25 is a schematic representation of a twelfth embodiment of an accessory for an intravenous infusion set; and

FIG. 26 is a schematic representation of the twelfth embodiment of an accessory for an intravenous infusion set.

FIG. 27 is a schematic representation of a thirteenth embodiment of an accessory for an intravenous infusion set;

FIG. 28 is a schematic representation of the thirteenth embodiment of an accessory for an intravenous infusion set;

FIG. 29 is a schematic representation of a fourteenth embodiment of an accessory for an intravenous infusion set;

FIG. 30 is a schematic representation of the fourteenth embodiment of an accessory for an intravenous infusion set;

FIG. 31 is a schematic representation of a fifteenth embodiment of an accessory for an intravenous infusion set;

FIG. 32 is a schematic representation of the fifteenth embodiment of an accessory for an intravenous infusion set;

FIG. 33 is a schematic representation of a sixteenth embodiment of an accessory for an intravenous infusion set;

FIG. 34 is a schematic representation of the sixteenth embodiment of an accessory for an intravenous infusion set;

FIG. 35 is a schematic representation of a seventeenth embodiment of an accessory for an intravenous infusion set;

FIG. 36 is a schematic representation of the primary engagement means of the seventeenth embodiment of an accessory for an intravenous infusion set;

FIG. 37 is a schematic representation of an eighteenth embodiment of an accessory for an intravenous infusion set;

FIG. 38 is a schematic representation of a nineteenth embodiment of an accessory for an intravenous infusion set;

FIG. 39 is a schematic representation of a twentieth embodiment of an accessory for an intravenous infusion set;

FIG. 40 is a schematic representation of a twenty-first embodiment of an accessory for an intravenous infusion set;

FIG. 41 is a schematic representation of a twenty-second embodiment of an accessory for an intravenous infusion set;

FIG. 42 is a schematic representation of a twenty-third embodiment of an accessory for an intravenous infusion set;

FIG. 43 is a schematic representation of the twenty-third embodiment of an accessory for an intravenous infusion set;

FIG. 44 is a schematic representation of a first embodiment of a swivel plate for an accessory for an intravenous infusion set;

FIG. 45 is a schematic representation of the first embodiment of the swivel plate, the twenty-third embodiment of an accessory for an intravenous infusion set, and a patient identification bracelet;

FIG. 46 is a schematic representation of the first embodiment of the swivel plate attached to the twenty-third embodiment which in turn is attached to a patient identification bracelet;

FIG. 47 is a schematic representation of a second embodiment of a swivel plate for an accessory for an intravenous infusion set;

FIG. 48 is a schematic representation of the second embodiment of a swivel plate attached to a patient identification bracelet;

FIG. 49 is a schematic representation of the second embodiment of a swivel plat, the twenty-third embodiment of an accessory for an intravenous infusion set, and a patient identification bracelet; and

FIG. 50 is a schematic representation of the second embodiment of the swivel plate attached to the twenty-third embodiment which in turn is attached to a patient identification bracelet.

### DETAILED DESCRIPTION OF THE DRAWINGS

With reference to the drawings in which like features are indicated by like numerals, accessory for securing an intravenous infusion set (IV set) to at least part of a user’s forearm is generally indicated by reference numeral 1.

The accessory 1 is described with reference to the embodiments thereof shown in the figures. All embodiments include primary engagement means 2 for engaging the accessory to a portion of a main intravenous line 3 (IV line) which forms part of an IV set 4. The IV line 3 is a central and main part of tubing of the set 4 which conveys fluids and medication from a bag (not shown) to a drip chamber and spike 8 and then to a Luer lock 7 with a cannula 11 attached which is inserted into a user’s vein.

The accessory 1 also includes secondary engagement means 5 for simultaneously engaging the intravenous line 3 to another portion along the line 3. The primary engagement means 2 is displaceable along the line 3 such the position of the accessory 1 may be adjusted along the length of the line 3 and encloses the periphery of the line 3. This ensures that when the primary engagement means 2 is engaged to the line 3 it is movable along the line 3 but not removable from the line 3 when in use. This is achieved in the embodiments by sizing the primary engagement means 2 to the size of the line 3. As the line 3 typically has connected accessories at either end thereof, this prevents the accessory 1 from being removed from the set 4 once installed.

In some embodiments the primary engagement means 2 engages the line 3 with a frictional fit such that the accessory 1 is movable along the line 3 when the friction of the engagement is overcome. This allows the engagement to be relatively stable against accidental or inadvertent movements and requires conscious effort to adjust.

The primary 2 and secondary 5 engagement means are proximate to one another such that, operatively, when the secondary engagement means 5 engages the line 3, the line 3 forms a loop 6. In some embodiments the primary 2 and secondary 5 engagement means are integrally formed.

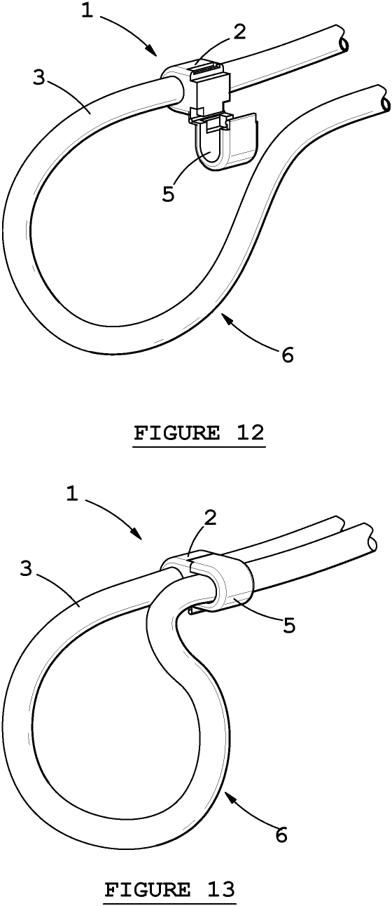

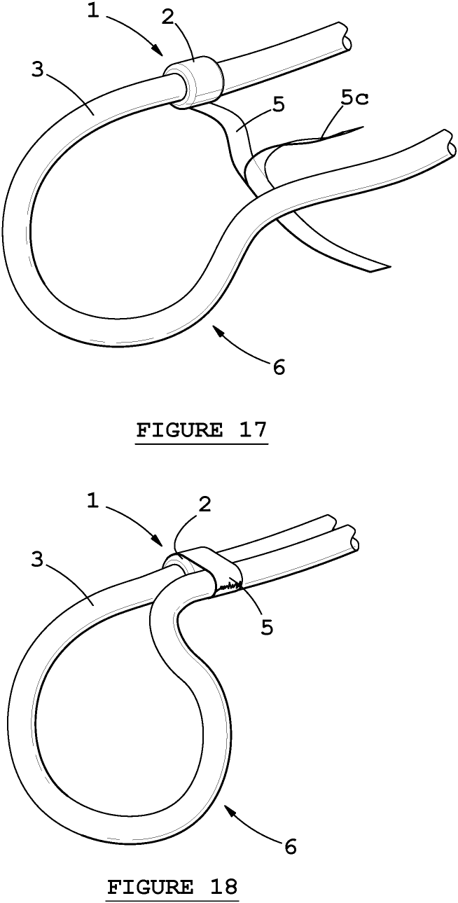

The primary engagement means 2 may be in the form of an aperture through the accessory, a tube, a sleeve, or a ring. The secondary engagement means 5 may be in the form of an aperture through the accessory 1, a tube, a sleeve, or a clip. Where the secondary engagement means 5 is in the form of a clip 5, as shown in FIGS. 12 and 13, the clip 5 may form a snap-fit engagement with the line 3. Similarly, the secondary engagement means 5 may be in the form of an adhesive strip 5, as shown in FIGS. 17, 18, and 19 which is secured to the primary engagement means 2. The adhesive strip 5 may take on many shapes to aid engagement of the accessory to the set 4 and may consist of various materials which allows securement.

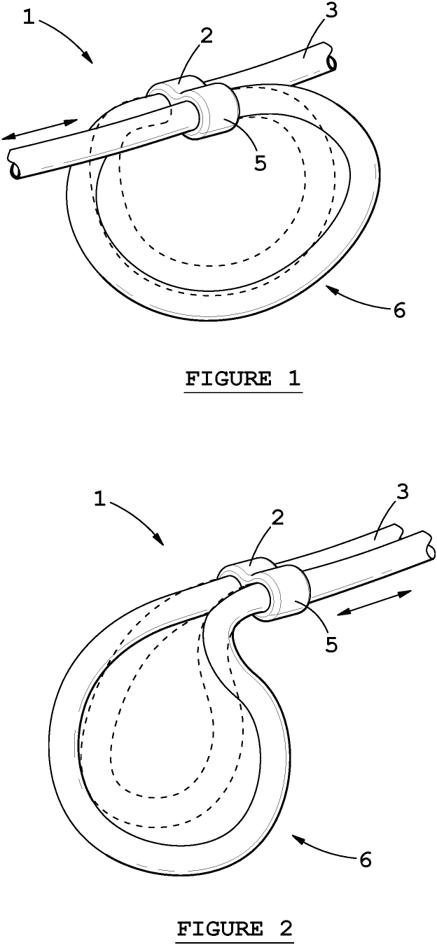

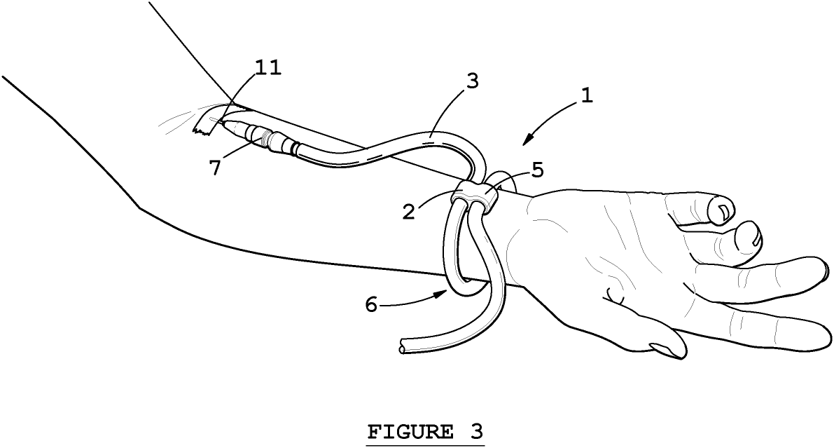

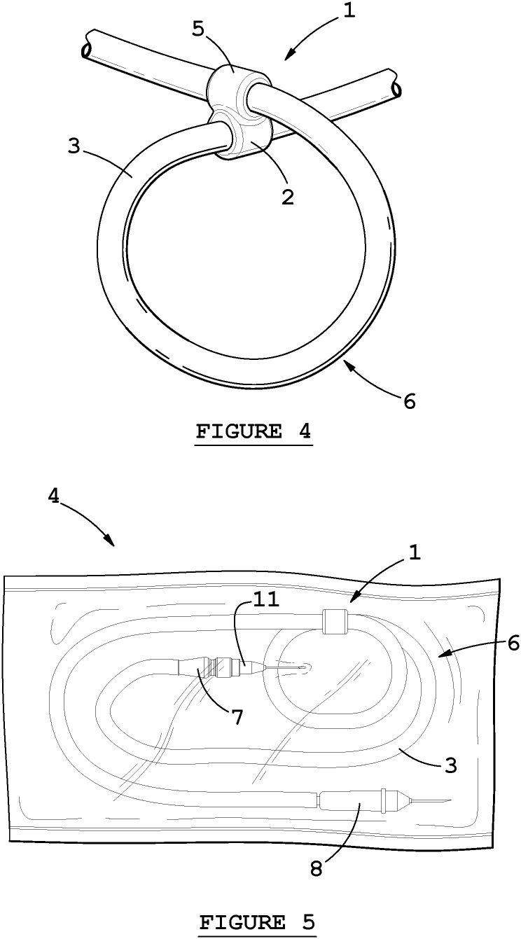

In the first embodiment, shown in FIGS. 1, 2, and 3 the accessory 1 has a substantially prismatic body with two parallel apertures extending through the body. In the second embodiment shown in FIG. 4 the accessory has two apertures facing in different directions rather than being parallel to each other. Each of these apertures may interchangeably serve as the primary 2 or secondary 5 engagement means of the accessory 1. Both apertures are sized to frictionally engage the line 3 and the accessory will typically be installed at the time of packaging the set 4.

In the first embodiment, when the set is packaged, the line 3 is threaded through one of the apertures and back through the other aperture to form a loop 6 in the line before additional accessories are added to the set 4. The loop 6 may either be in the form shown in FIG. 2 where the direction of threading is the same and the loop 6 is formed about the accessory. Alternatively, the direction of threading may be reversed, thereby forming the loop 6 externally to the accessory as shown in FIG. 1. This loop may be placed about an arm or wrist of the user and the loop may be tightened to secure the line to the user. Once additional accessories are added to the set 4, it is not possible to remove the accessory from the line 3. This will encourage medical staff to secure the line to the user’s arm during set up.

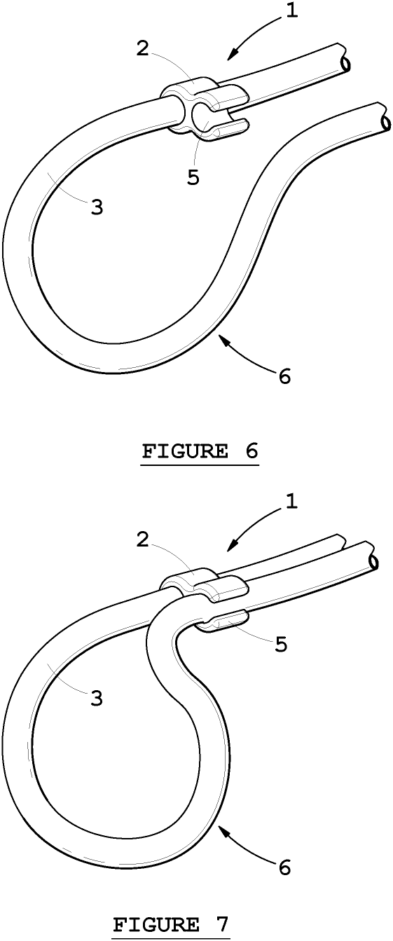

A third embodiment is shown in FIGS. 6 and 7. In this embodiment, the accessory 1 has primary engagement means which is similar to the first embodiment described above. The secondary engagement means 5 takes the form of a clip which is integrally formed as a channel which runs parallel to the aperture of the primary engagement means 2. The clip is shaped and sized to releasable engage the line 3 in a snap fit configuration. This allows the loop 6 to be formed or released by engaging and disengaging the line to the clip.

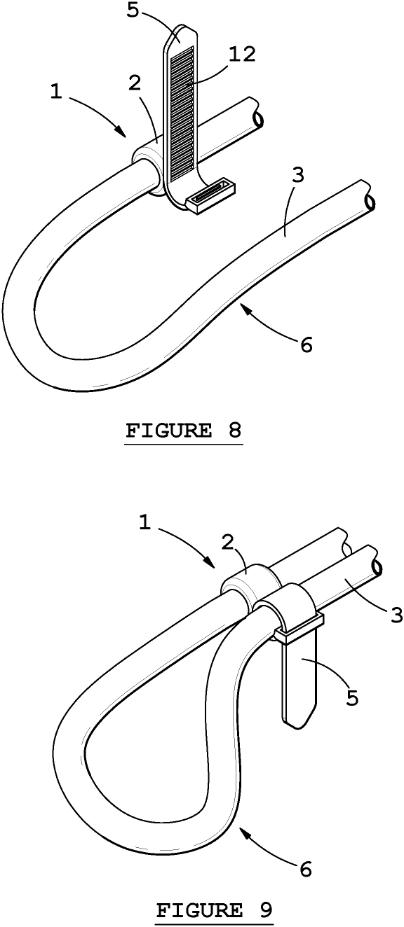

A fourth embodiment is shown in FIGS. 8 and 9. In this embodiment, the secondary engagement means 5 which is wider than the circumference of the line 3 and includes frictional adjustment means such as a serrated inside 12. This allows the secondary engagement means 5 to be constricted to fit the size of the IV line 3 and to be kept in place by the serrated inside as shown in FIG. 9.

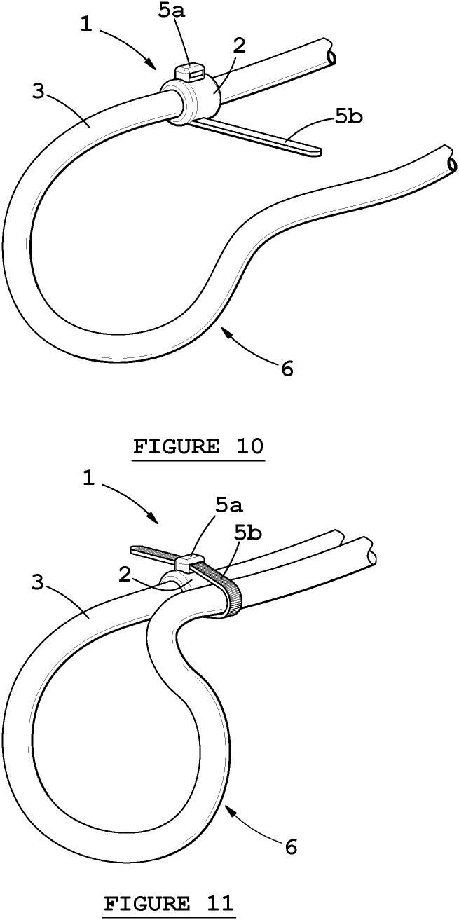

A fifth embodiment is shown in FIGS. 10 and 11 wherein the primary engagement means 2 is in the form of a tube 2. The line 3 may be threaded through the tube 2 at the time of packaging the set 4. In this embodiment, the secondary engagement means 5 is in the form of a releasable clamp 5 with two halves of the clamp (5a and 5b) movable relative to each other (and relative to the tube) in a hinged fashion. The halves are movable between an open position, wherein the line 3 may be inserted for engagement and a closed position, wherein the line therein is engaged. The clamp 5 may maintain the halves in the closed position by integrally formed snap-fit formations on each half or external fasteners such as screws, locks, or clips may be employed to maintain the halves in the closed position. The clamp 5 has integrally formed channels in either half which extend along the length of the clamp parallel to the tube and, when closed, forms an aperture which engages the line 3.

A sixth embodiment is shown in FIGS. 12 and 13 wherein the primary engagement means is a ring which is engaged to the line. The secondary engagement means 5 in this embodiment is in the form of a hook shaped latch 5 which pivots relative to the ring 2. The secondary engagement means 5 may also form a latch 5 which swivels relative to the ring 2. The ring 2 is installed about the tube at the time of packaging. During set up, the line 3 may be looped about the arm of the user and the loop may be secured in place by hooking the latch about the line 3 after the loop is formed.

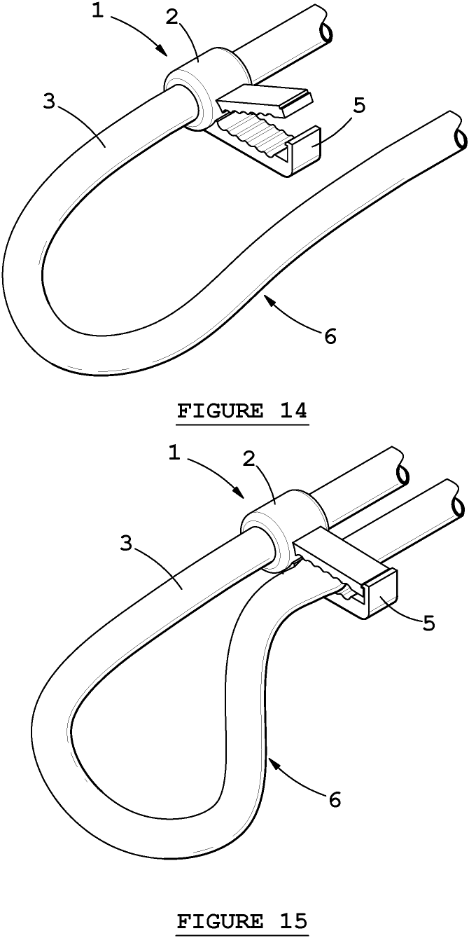

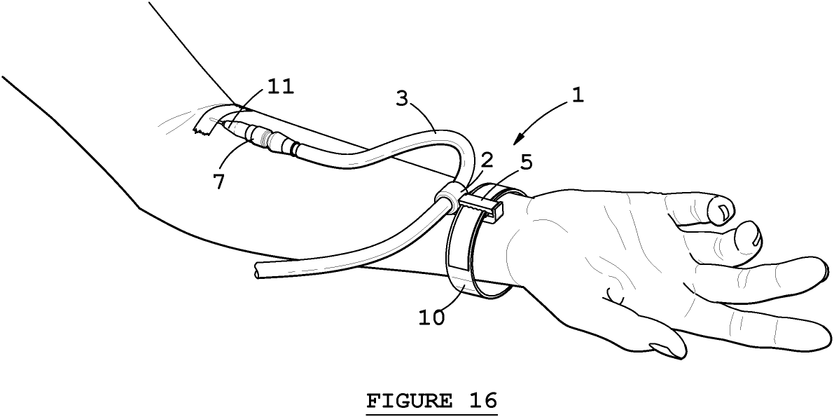

A seventh embodiment is shown in FIGS. 14, 15, and 16. In this embodiment, the accessory 1 has primary engagement means 2 which is similar to the first embodiment described above. The secondary engagement means 5 may be a clip, hook, or clamping mechanism 5 which may be attached to the IV line 3 to form a loop 6 as shown in FIG. 15. Alternatively, the clamping mechanism 5 may be attached to a user’s identification bracelet 10 as shown in FIG. 16. This eliminates the need to form a loop 6 and also allows the accessory to be simultaneously attached to the line 3 and the user’s identification bracelet 10.

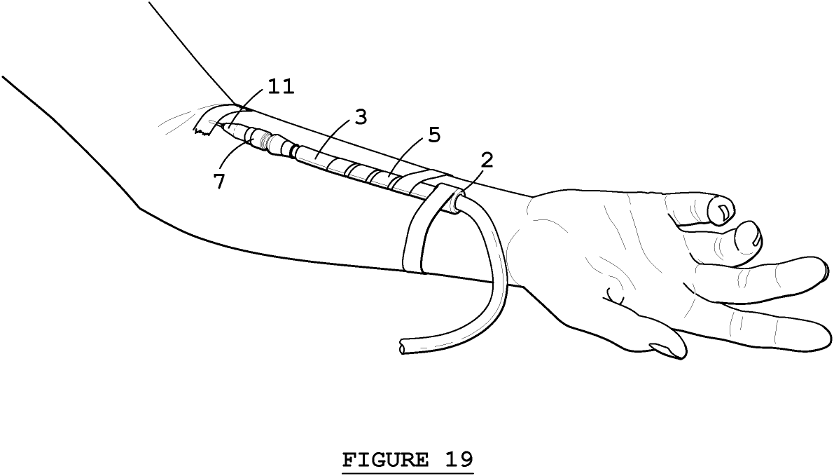

An eight embodiment is shown in FIGS. 17, 18, and 19. In this embodiment the primary engagement means 2 is similar to the first embodiment described above. Integrally formed with the primary engagement means 2 is an elongated strip of adhesive material 5. The adhesive material 5 may include a backing strip 5c which may be removed when used. In this embodiment, the accessory will again typically be installed during packaging. When used, the line may be formed into a loop 6 which is placed about the arm of a user. The backing strip 5c may then be removed from the adhesive strip in order to engage the line 3 and to secure the loop 6 about a user’s wrist or arm. An alternative form of the adhesive strip is shown in FIG. 19, which is an extension of the current embodiment. Herein the adhesive strip 5 is extended to wrap around the user’s forearm before also wrapping around the IV line 3. This eliminates the need to form a loop 6 and also allows the accessory 1 to be simultaneously attached to the line 3 and the user.

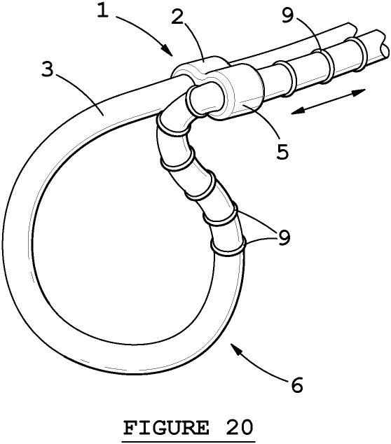

A ninth embodiment of the accessory 1 is shown in FIG. 20. In this embodiment the primary 2 and secondary 5 engagement means are similar to the first embodiment however, the IV line has integrally formed circumferential protrusions 9. This allows the secondary engagement means 5 to be clipped over the protrusions 9 in order to secure the accessory 1.

A tenth embodiment of the accessory is shown in FIGS. 21 and 22. In this embodiment the secondary engagement means 5 are integrally formed protrusions 5 extending from the IV line 3. The accessory 1 may be looped around the user’s forearm and the primary engagement means 2 may be clipped over the desired protrusion 5 to form a loop 6 which is a suitable size for the user’s forearm.

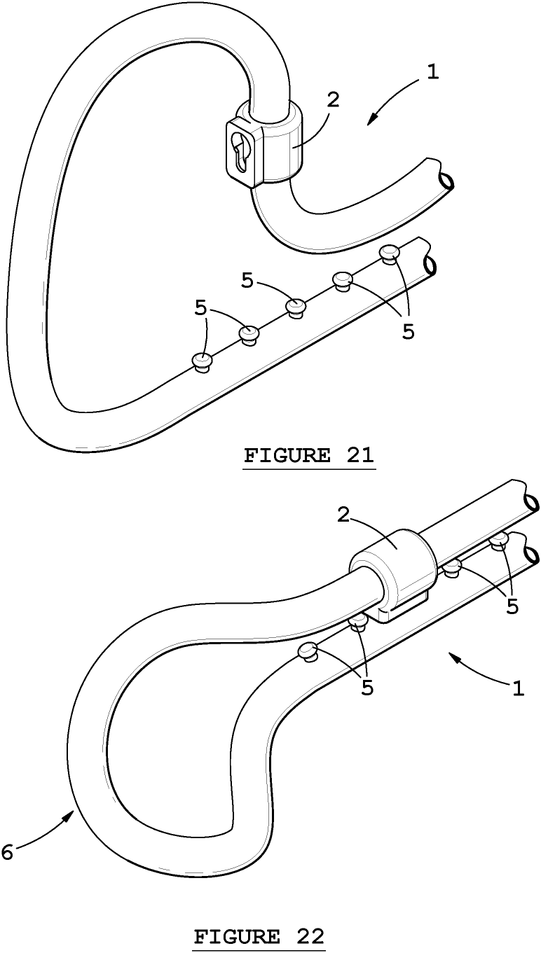

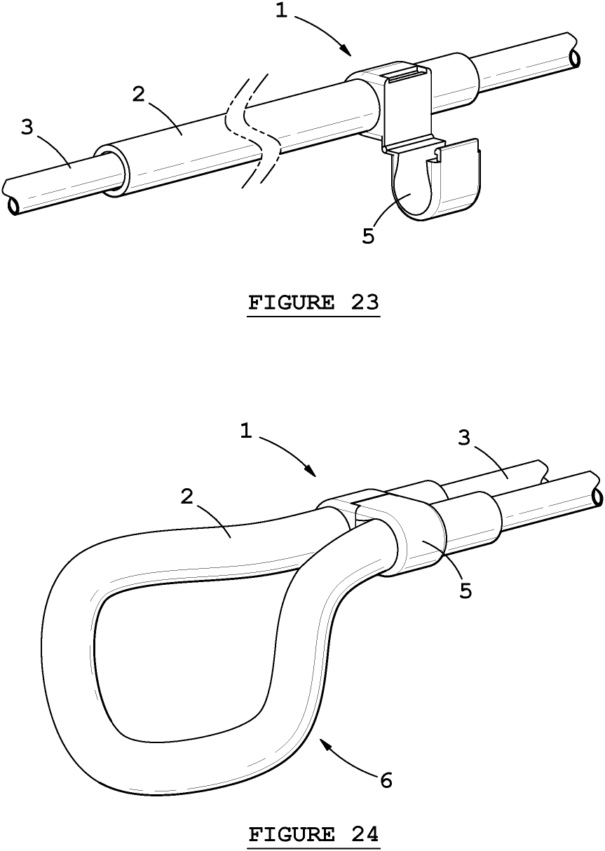

An eleventh embodiment of the accessory is shown in FIGS. 23 and 24. In this embodiment the primary engagement means 2 is a sleeve 2 which covers the IV line 3. The IV line 3 is twisted to form a loop and the sleeve 2 is secured in the loop formation by the secondary engagement means 5 which may be a clamping mechanism 5.

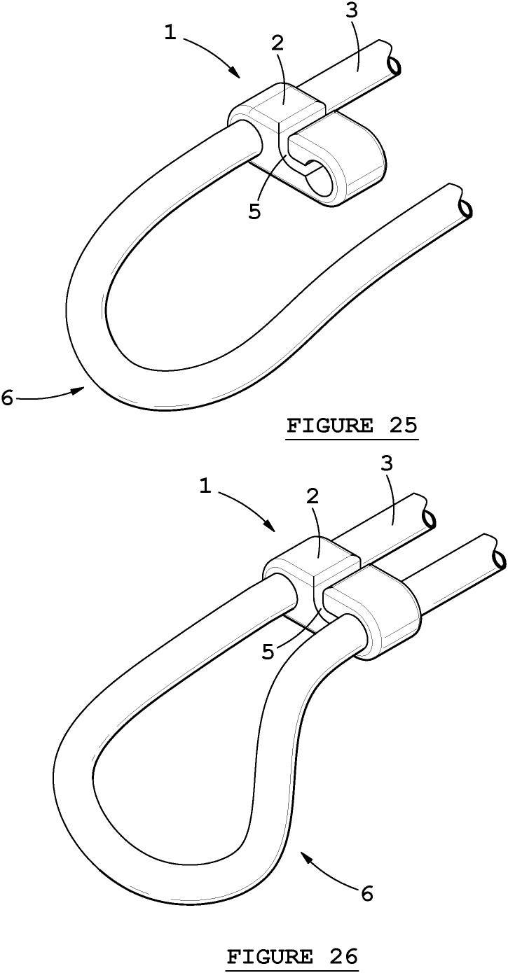

A twelfth embodiment of the accessory 1 is shown in FIGS. 25 and 26. In this embodiment, the accessory 1 has primary engagement means 2 which is similar to the first embodiment described above. The secondary engagement means 5 takes the form of an entry slit 5 in which a second part of the IV line 3 is slid into and contained in an aperture next to the aperture of the primary engagement means 2. The slit 5 is shaped and sized to releasable engage the line 3 in a snap fit configuration. This allows the loop 6 to be formed or released by engaging and disengaging the line to through the slit 5.

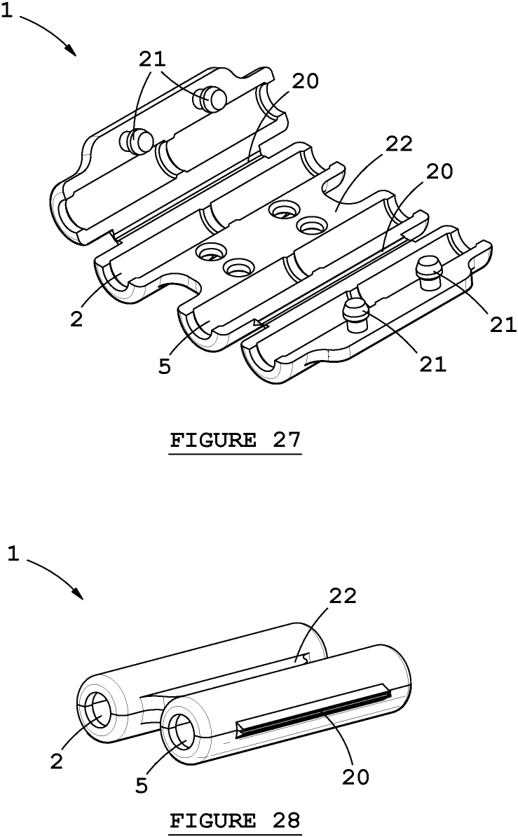

A thirteenth embodiment of the accessory 1 is shown in FIGS. 27 and 28. In this embodiment the accessory 1 has a primary engagement means 2 and a secondary engagement means 5 which are interchangeable and integrally formed. The accessory includes a center portion 22 between the primary engagement means 2 and the secondary engagement means. An IV line (not shown) is receivable when the accessory 1 is in the open position as shown in FIG. 27. The IV line is then retained inside the accessory once in the closed position as shown in FIG. 28. The accessory 1 has two hinged sides 20 with protrusions 21 which folds over onto the center 22 of the accessory 1 and clips the hinged sides 20 to the center 22 of the accessory 1.

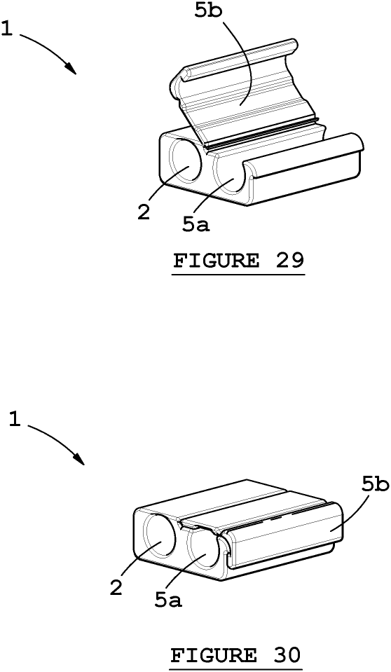

A fourteenth embodiment of the accessory 1 is shown in FIGS. 29 and 30. In this embodiment the primary engagement means 2 are similar to the first embodiment described above however, the primary engagement means are substantially rectangular on the outside with a circle shaped aperture inside for threading the IV line through. The secondary engagement means 5 in this embodiment is in the form of a receiving part 5a integrally formed with the primary engagement means 2 and a latch 5b which pivots relative to the receiving part 5a. The latch 5b may also swivel relative to the receiving part 5a and be locked in place. The accessory 1 is installed about the tube at the time of packaging. During set up, the IV line may be looped about the arm of the user and the loop may be secured in place by locking the latch 5b about the line after the loop is formed.

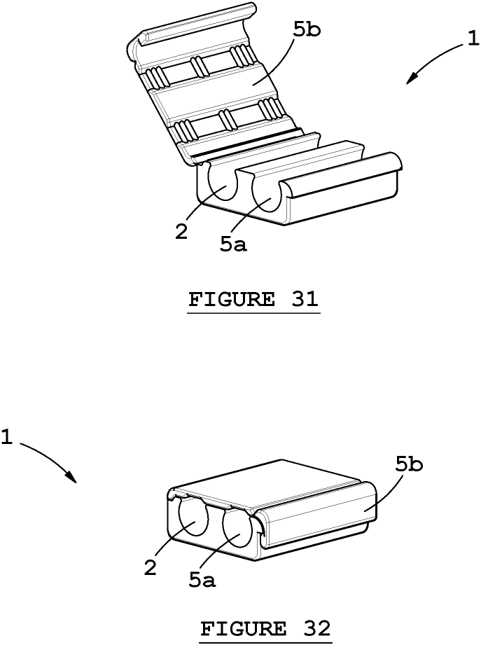

A fifteenth embodiment of the accessory 1 is shown in FIGS. 31 and 32. This embodiment is similar to the fourteenth embodiment discussed above however, the primary engagement means 2 is in the form of a receiving part 2a similar to the secondary engagement means receiving part 5a. The latch 5b is attached to the receiving part 2a of the accessory 1. The latch 5b swivels from an open position as shown in FIG. 31 to a closed position as shown in FIG. 32. The latch 5b encloses both the primary and secondary receiving parts 2a and 3a.

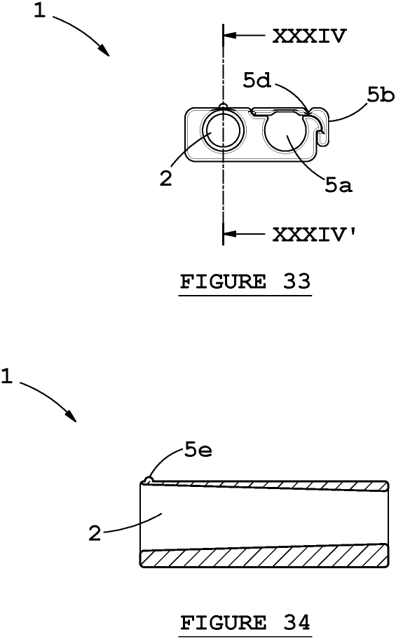

A sixteenth embodiment of the accessory 1 is shown in FIGS. 33 and 34. In this embodiment the primary engagement means 2 are similar to the fourteenth embodiment however the aperture inside the primary engagement means 2 is tapered in order to retain the IV line using a friction fit. The latch 5b includes a v shaped groove 5d which serves as a weakened portion of the latch 5b. In use, the latch 5b will break at the groove 5d if the latch 5b is reopened continuously by, for instance, a healthcare worker. This feature prevents use of the same accessory 1 on different patients. FIG. 34 more clearly shows the tapering of the aperture which will result in a circumference fit of the IV line. The protrusion 5e shown in FIG. 34 serves as an indicator for factory assemblers to rapidly determine from which end of the accessory 1 the IV line should be threaded through.

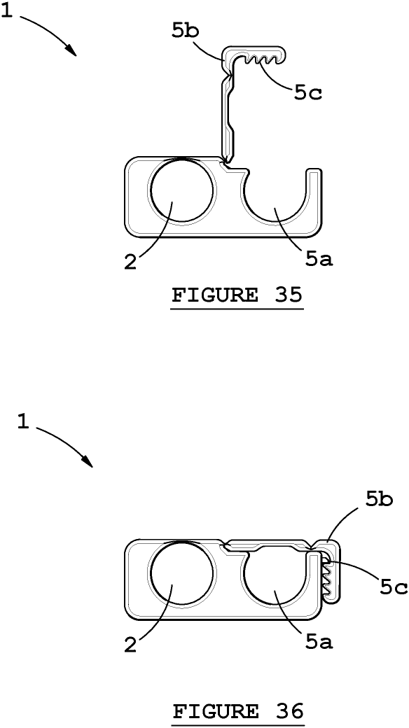

A seventeenth embodiment of the accessory 1 is shown in FIGS. 35 and 36. The embodiment shown in these figures are similar to the fourteenth embodiment however the latch 5b has various hooks 5c which locks onto the receiving part 5a. In use, the latch can be adjusted to make provision for larger or smaller IV lines or medical tubes.

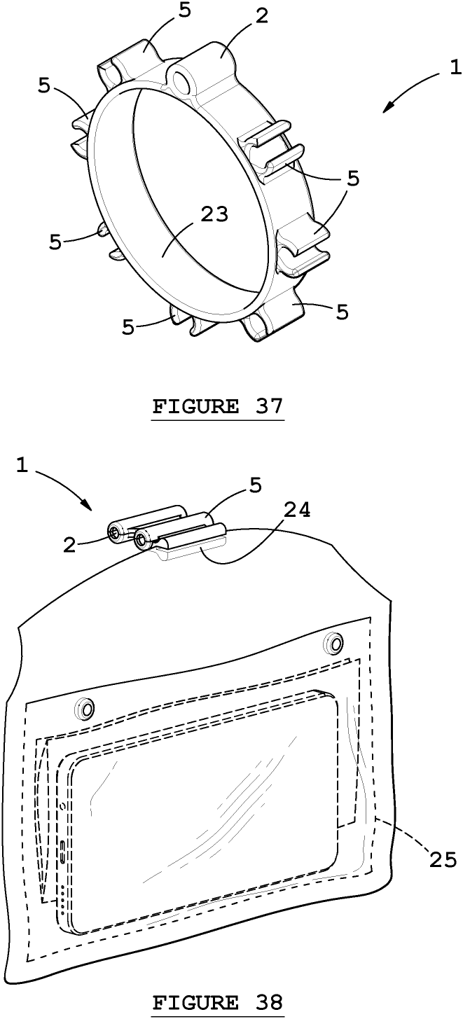

An eighteenth embodiment of the accessory 1 is shown in FIG. 37. In this embodiment the accessory 1 has primary engagement means 2 which is similar to the first embodiment described above. The secondary engagement means 5 are in turn similar to the secondary engagement means 5 described in the third embodiment however, instead of the accessory 1 only having a single secondary engagement means 5, it has multiple secondary engagement means 5. The multiple secondary engagement means 5 takes the form of clips which are integrally formed to the body 23 of the accessory 1. The primary engagement means 2 are also integrally formed to body 23 of the accessory 1. The clips 5 are shaped and sized to releasable engage a line in a snap fit configuration.

A nineteenth embodiment of the accessory 1 is shown in FIG. 38. In this embodiment the accessory 1 is similar to the thirteenth embodiment described above. The accessory 1 however has an additional clip 24 integrally formed to the primary and secondary engagement means 2 and 5. In this embodiment a pouch 25 is retained by the clip 24. The pouch 25 may contain a patient’s valuables, medication, or medical documentation. The contents of the pouch 25 are easily accessible due to the pouch being attached to the accessory 1.

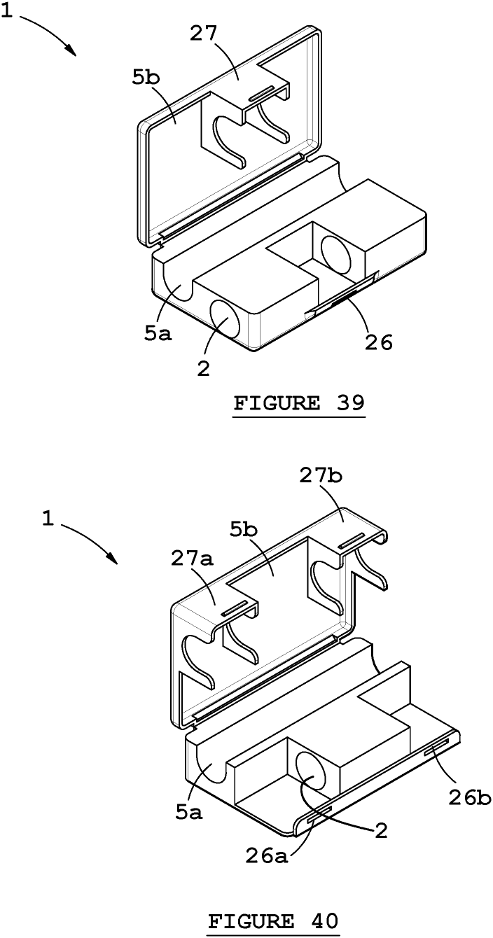

A twentieth embodiment of the accessory 1 is shown in FIG. 39. In this embodiment the primary engagement means 2 includes a clip 26 and an open section in the center of the primary engagement means 2. The open section receives a clawed clamp 27 attached to the latch 5b. The latch 5b is pivotally attached to the receiving part of the secondary engagement means 5a. The IV line is threaded through the primary engagement 2 means when being assembled in a factory such that the IV line is non-releasable from the accessory 1. In use, a second part of the IV line is placed in the receiving part 5a, the latch 5b is closed, and the clawed clamp 27 engages the clip 26 locking the accessory 1 into a closed position. The curved shape of the clawed clamp 27 frictionally engages the IV line when closed and keeps the IV line in place.

A twenty-first embodiment of the accessory 1 is shown in FIG. 40. In this embodiment the accessory 1 is similar to the twentieth embodiment however, the accessory 1 includes two clips, 26a and 26b, as well as two clawed clamps, 27a and 27b, located on the ends of the accessory 1.

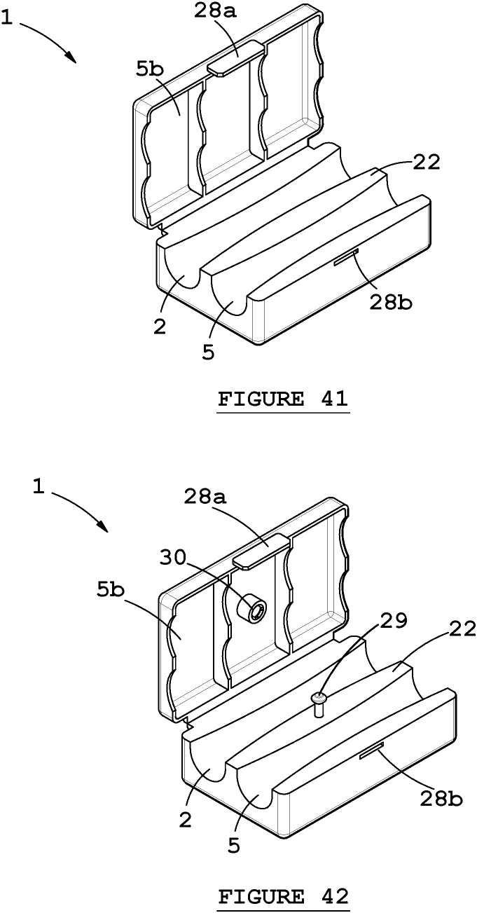

A twenty-second embodiment of the accessory 1 is shown in FIG. 41. In this embodiment the primary engagement means 2 and the secondary engagement means 5 are shaped to form concaved inner receiving parts. The narrowed sections of the concaved inner receiving parts frictionally engage the IV line. If the IV line is pulled it will not slide through the holes due to the frictional engagement. The accessory 1 includes a center portion 22 between the primary engagement means 2 and the secondary engagement means 5. The primary engaging means 2 includes a latch 5b which is pivotally attached to the primary engagement means 2. The latch 5b and the secondary engagement means 5 includes a locking mechanism 28a and 28b for securing the accessory in a closed position.

The concaved inner receiving parts also reduces kinking of the IV line while still allowing movement of the lines in a lateral direction. Kinking occurs when the IV line is bent too far against the edges of the accessory 1 which occludes the lumen and therefore prevents flow of fluid through the IV line.

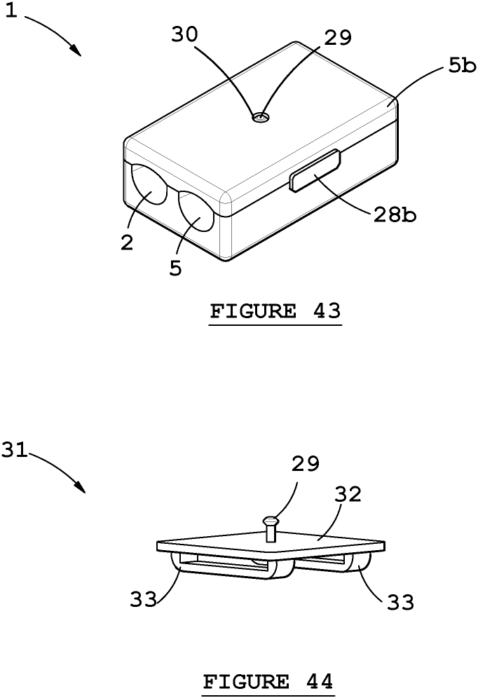

A twenty-third embodiment of the accessory 1 is shown in FIG. 42. This embodiment is substantially similar to the twenty-second embodiment however, in this embodiment the accessory 1 includes a spike or pin 29 on the center portion 22 of the accessory and a corresponding hole 30 for receiving the pin 29. A person skilled in the art will appreciate that the spike 29 and hole 30 may be substituted by a different engaging formation which still serves the same function of engaging the latch 5b with a portion of the engagement means 2 and 5. In use, a first part of the IV line is placed in the receiving part of the primary engagement means 2, the latch 5b covers the primary 2 engagement means, and the spike 29 engages the hole 30 in the latch 5b. The spike may be long enough to allow a second part of the IV line to be inserted and/or removed from the secondary engagement means 5 when the spike 29 and hole 30 is engaged. The latch 5b is then closed further and the locking mechanism 28a and 28b locks the accessory into a closed position. The pin 29 which is pressed through the hole 30 further secures the accessory 1 in a closed position, allows access to the secondary engagement means receiving part 5, and restricts access to the primary engagement means receiving part 2. The pin 29 and hole 30 engages in a snap fit configuration and fastens the accessory such that the latch 5b is non-releasable from the primary 2 and secondary 5 engagement means as shown in FIG. 43. If a healthcare worker attempts to open the locking mechanism 28a and 28b, the pin 29 will deform. The deformation of the pin 29 renders the accessory 1 unusable. This ensures that the accessory 1 is only used once and not on multiple patients. The accessory may also include more than one pin and hole (not shown) to further ensure a tamper proof accessory.

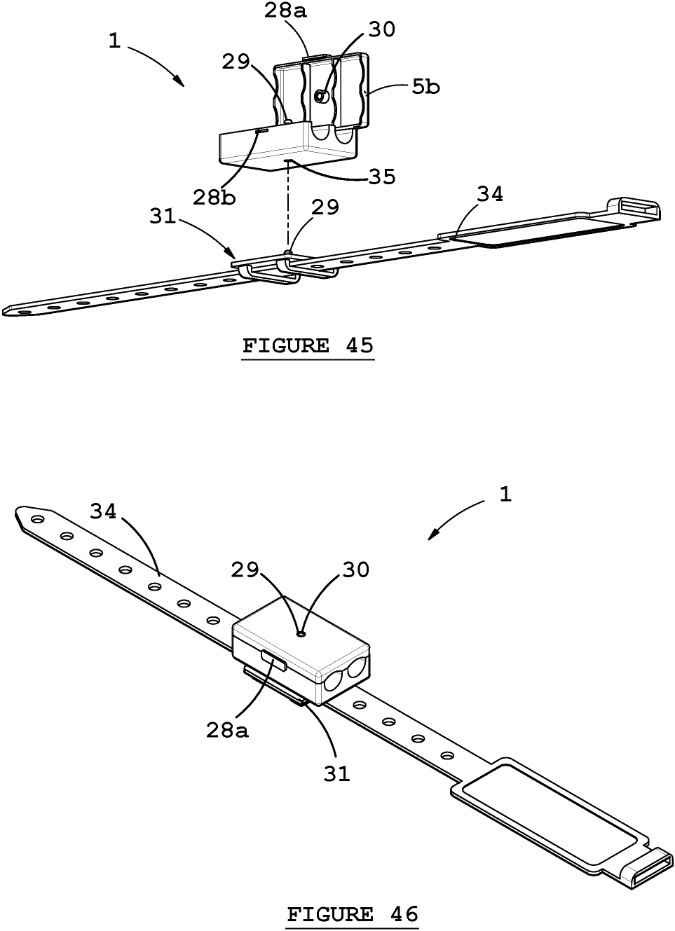

FIG. 44 shows a first embodiment of an additional accessory 31 which may be used in conjunction with an accessory 1 as described above. The additional accessory 31 comprises a plate 32, a spike 29 on a first side of the plate 32 and one or more handles 33 on a second side of the plate 32. The handles 33 creates a slot for sliding a patient’s identification bracelet through. As shown in FIGS. 45 and 46, the additional accessory 31 is used in conjunction with the twenty-third embodiment of the accessory 1. In this instance the twenty-third embodiment of the accessory 1 includes a hole 35 at the bottom for receiving the spike 29 from the additional accessory 31. An identification bracelet 34 slides through the slots created by the handles 33 and the accessory 1 is non-releasably clipped onto the additional accessory 31 using the snap fit configuration of the spike 29 and hole 35. The additional accessory 31 serves as a swivel plate which allows the accessory 1 to rotate relative to the bracelet 34 and the patient’s arm. The rotation feature further reduces the possibility of kinking of the IV line.

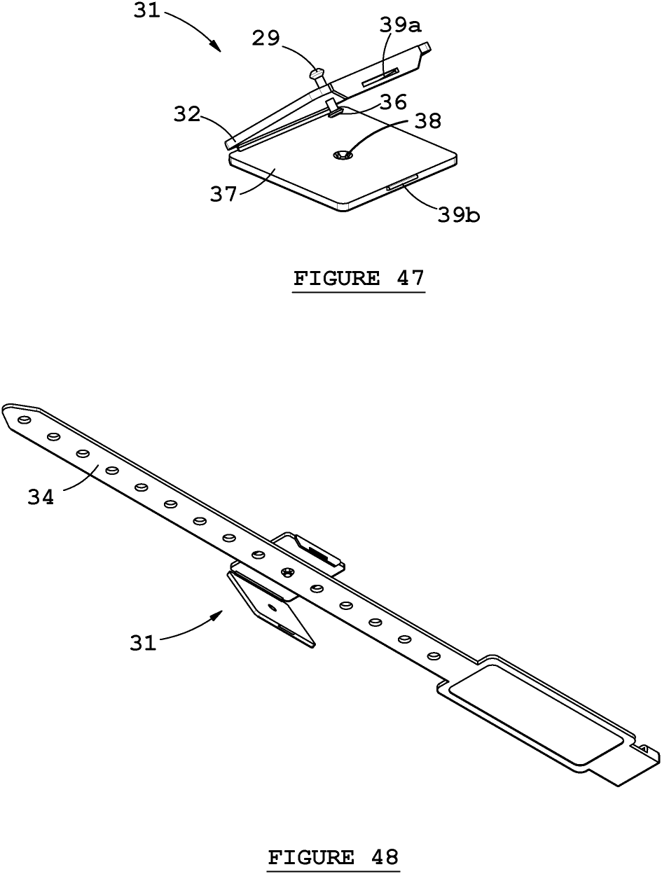

A second embodiment of the additional accessory or swivel accessory 31 is shown in FIG. 47. In this embodiment the accessory 31 includes a plate 32, a spike 29 on a first side of the plate 32 and another spike 36 on a second side of the plate 32. The accessory 31 also includes a second plate 37 pivotally attached to the first plate 32. The second plate includes a hole 38 for receiving the spike 36 on the bottom of the first plate 32. The plates also include a locking mechanism 39a and 39b which, when locked, prevents the plates from being separated and locked again. This feature prevents the additional accessory 31 from being reused. As shown in FIG. 48, the accessory 31 is attached to an identification bracelet by pressing the spike 36 through the holes in the identification bracelet and into the hole 38 of the second plate 37. The two plates are locked together and similar to the functionality describe in the twenty-third embodiment, the spike 36 and hole 38 non-releasably attaches the accessory 31 to the identification bracelet 34.

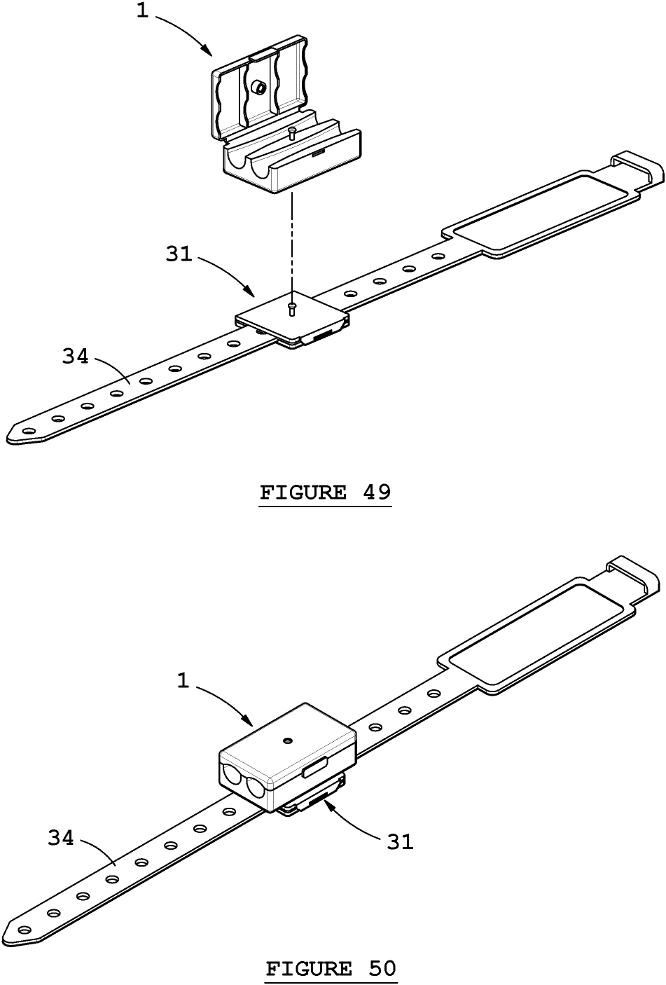

FIGS. 49 and 50 show the twenty-third embodiment of the accessory 1, the second embodiment of the swivel accessory 31, and the identification bracelet 34 in use. The swivel accessory 31 is non-releasably attached to the bracelet 34 after which the accessory 1 is non-releasably attached to the swivel accessory 31.

The accessory 1 may also include embodiments wherein the additional accessories described above are integrally formed with the body of the accessory 1. The accessory 1 may be manufactured to include means for attaching the accessory directly to the identification bracelet without the need for an additional accessory. The accessory 1 may include handles 33 as described in FIG. 44 which are not separate from the body of the accessory 1. The handles 33 which creates a slot for the identification bracelet 34 to be inserted may also be a clip, hook, or clamping mechanism as described in FIGS. 14 to 16 as well as FIG. 38 which shows the additional clip attached to the secondary engagement means 5. The accessory may also be attached to the identification bracelet with a spike as described in FIGS. 47 and 49 however, the spike or pin is manufactured to be attached directly to the body of the accessory 1. In another embodiment not shown herein, the accessory 1 may include a lid, flap, or latch formation for retainment to the identification bracelet.

The invention also comprises an IV set 4 which may be prepacked for use as shown in FIG. 5. The set 4 includes a tubular main line 3, a drip chamber and spike 8 at one end of the main line 3, and a Luer lock 7 at the other end of the main line. The set 4 includes a loop 6 in the main line 3 (the main line 3 bends around and crosses itself to form the loop 6) and an accessory 1 which retains the loop 6 in the main line 3 such that the line may be moved relative to the accessory 1 to adjust the dimensions of the loop 6. When packaged as an IV set 4, the accessory 1 may take the form of any of the embodiments described herein or may take on any other form which engages both line portions and allows relative movement between the portions engaged by the line. In use, the set will be removed from the packaging by a nurse and set up such that the loop 6 engages at least part of a patient’s forearm and movement of the line 3 between the drip chamber and spike 8 and the loop 6 will be imparted to the patient’s arm and the movement will be prevented from being imparted on the Luer lock 7 of the IV set 4.

The invention also extends to a method of setting up an intravenous set 4. The method will typically be performed by a nurse or other medical staff during set up of the IV set 3. The method includes the steps of providing an IV set with a tubular main line 3, a distal end 8, and a proximal end 7. The set will typically be provided in the form of a sterile pre-packaged set 4 as shown in FIG. 4. The method includes the step of forming a loop 6 in the main line 3 between the proximal 7 and distal 8 ends of the line 3. This step will either be performed by the nurse or when the set 4 is pre-packaged. The following step, wherein the loop 6 is engaged to at least part of the patient’s forearm, is also performed by a nurse and once the loop 6 is engaged, it may be secured to maintain the loop 6 about the patient’s forearm. Securing may be done by fixing the two portions of the line 3 to each other (using for example adhesive tape) or it may be done by using any embodiment of accessory 1 described above.

It is envisaged that the invention will provide simple, cost effective, and highly effective means of preventing a cannula from accidentally or inadvertently being removed from a patient. The simplicity of the invention makes it highly suitable for high volume manufacturing such that it will add very little additional expense to the end user.

The invention is not limited to the precise details as described herein. For example, instead of having parallel apertures, the apertures may form an angle between them. Further, the examples above are described with reference to intravenous infusion, but those skilled in the relevant art will appreciate that the invention will be just as effective and useful in, for example, arterial infusion or blood transfusions which has very similar equipment. Further, all the examples described herein make use of single primary and secondary engagement means but having tertiary engagement or dual secondary engagement may be beneficial in some circumstances. The embodiments of the accessory described herein is not limited to a non-releasable state once closed and a person skilled in the art will appreciate that the accessory may include engaging mechanisms which will not deform when opened and still allow the accessory to be used more than once. The additional accessories described herein may also either be non-releasable or releasable to the main accessory and the identification bracelet.

1. An accessory for securing an intravenous infusion set to at least part of a user’s forearm, the accessory comprising:
primary engagement means for frictionally engaging the accessory to a portion of a main intravenous line of the set, the primary engagement means including an aperture that is sized and configured to operatively enclose a periphery of the portion of the main intravenous line that is being engaged;
the primary engagement means being displaceable along the length of the main intravenous line, whilst the aperture encloses a periphery of the main intravenous line and between additional accessories that are connected to the main intravenous line, when the friction of engagement is overcome; and
secondary engagement means for simultaneously engaging the accessory to another portion of the main intravenous line;
the primary and secondary engagement means being proximate to one another such that, operatively, when the secondary engagement means also engages the line, the main intravenous line forms a loop;
wherein, in use, the loop is placed about the forearm, or a wrist of the user, and tightened to secure the intravenous infusion set to the user’s forearm by displacing the primary engagement means along the length of the main intravenous line to adjust the dimension of the loop.

2. The accessory of claim 1 wherein the secondary engagement means frictionally engages the main intravenous line.

3. The accessory of claim 1, wherein the primary engagement means comprise a tube that includes the aperture, a sleeve that includes the aperture, or a ring that includes the aperture.

4-6. (canceled)

7. The accessory of claim 1, wherein the secondary engagement means is a tube, a sleeve, or an adhesive strip.

8. (canceled)

9. The accessory of claim 1, wherein the secondary engagement means is a clip.

10. The accessory of claim 9, wherein the clip forms a snap-fit engagement with the main intravenous line.

11-14. (canceled)

15. The accessory of claim 1, wherein the secondary engagement means includes a tag for writing on or attaching a preprinted label.

16. The accessory of claim 1, wherein the primary and secondary engagement means are parallel to each other to maintain the engaged portions of the main intravenous line parallel to each other.

17. The accessory of claim 1, wherein the primary and secondary engagement means are at an angle to each other to maintain an angle between the engaged portions of the main intravenous line.

18. The accessory of claim 1, wherein the primary and secondary engagement means are integrally formed.

19. The accessory of claim 1, wherein the primary and secondary engagement means are pivotally attached to one another such that a hinged connection is formed.

20. The accessory of claim 1, wherein the secondary engagement means includes an accessory attachment means for attaching additional accessories to the accessory.

21. The accessory of claim 1, wherein the accessory further comprises an attachment means for attaching the accessory directly to an identification bracelet of the user, the attachment means being integrally formed with a body of the accessory and taking the form of handles that define a slot for receiving the identification bracelet, a clip, a hook, a clamping mechanism, a spike, or a pin.

22. The accessory of claim 1 wherein the primary engagement means includes adjustable line restricting means for adjustably restricting the flow of fluid through the engaged portion of the main intravenous line.

23. The accessory of claim 1 wherein the secondary engagement means includes adjustable line restricting means for adjustably restricting the flow of fluid through the engaged portion of the main intravenous line.

24. An intravenous infusion set comprising:
a tubular main line;
a catheter engagement mechanism at one end of the main line;
a drip chamber at the other end of the main line;
a loop in the main line wherein the main line bends around and crosses itself to form the loop; and
an accessory of claim 1 which retains the loop in the main line such that the line is displaced relative to the accessory to adjust a dimension of the loop,
wherein, in use, the loop engages at least part of a patient’s forearm such that movement of the line between the drip chamber and the loop is imparted to the patient’s forearm and prevents the movement from being imparted on the catheter engagement end of the intravenous infusion set.

25-29. (canceled)

30. A method of setting up an intravenous infusion set, the method including the steps of:
providing an intravenous infusion set with a tubular main line having a distal end and a proximal end;
providing an accessory according to claim 1;
forming a loop in the main line between the proximal and distal ends of the line;
engaging the loop to at least part of a patient’s forearm; and
securing the loop to maintain the loop about the patient’s forearm by engaging the primary engagement means of the accessory to a portion of the line and engaging the secondary engagement means of the accessory to another portion of the main line.

31. (canceled)

32. The accessory of claim 1, wherein the apertures of the primary and secondary engagement means are formed by respective curved receiving parts with a center portion of the accessory therebetween and a latch that is attached to the primary engagement means, the latch being configured to swivel from an open position wherein respective portions of the main intravenous line are insertable into the receiving parts and a closed position wherein the latch covers both receiving parts and operatively encloses a periphery of the portions of the main intravenous line to engage said portions.

33. The accessory of claim 32, wherein the secondary engagement means and the latch includes a locking mechanism for securing the latch in the closed position.

34. The accessory of claim 32, wherein a spike or pin extends from the center portion of the accessory and wherein the latch includes a hole for receiving and engaging the spike or pin to secure the latch in the closed position, the spike or pin having a length that permits it to engage the hole when the latch is swivelled to cover the receiving formation of the primary engagement means, only.
